(19) United States
(12) Patent Application Publication
(10) Pub. No.: US 2022/0233463 A1
(43) Pub. Date: Jul. 28, 2022

(54) LOCAL ANESTHETIC COMPRISING A TRP CHANNEL MODULATOR

(71) Applicant: BUZZELET DEVELOPMENT AND TECHNOLOGIES LTD., OR-AKIVA (IL)

(72) Inventors: NOA RAZ, GIZO (IL); AHARON EYAL, JERUSALEM (IL)

(21) Appl. No.: 17/611,583

(22) PCT Filed: May 14, 2020
(86) PCT No.: PCT/IB2020/054565

§ 371 Date: Nov. 16, 2021

Related U.S. Application Data
(60) Provisional application No. 62/958,911, filed on Jan. 9, 2020.
(60) Provisional application No. 62/848,589, filed on May 16, 2019.

(51) Int. Cl. A61K 31/05; A61P 29/00; A61K 31/352; A61K 31/045; A61K 31/365; A61K 31/125; A61K 31/245; A61K 31/445; A61P 23/02; A61K 31/11; A61K 31/085

(52) U.S. Cl. CPC A61K 31/05; A61P 29/00; A61K 31/352; A61K 31/045; A61K 31/365; A61K 31/085; A61K 31/245; A61K 31/445; A61P 23/02; A61K 31/11; A61K 31/125

(57) ABSTRACT

Provided are methods and compositions for reducing a throat irritation caused by inhaling vapors or particles of at least one material selected from the group consisting of cannabis, cannabinoids, tobaccO, a herb or combinations thereof, the method comprising administering to a subject in need thereof at least one transient receptor potential (TRP) channel modulator prior to or simultaneously with said inhaling said vapors or particles of said at least one material. Further provided are methods and compositions comprising at least one TRP channel agonist, and/or at least one TRP channel partial agonist for treating pain-selective analgesia via TRP channels; methods for treating local pain; methods for treating migraine and symptoms thereof; and methods for treating osteoporosis and symptoms thereof.

### RELATED APPLICATIONS

The present application claims the benefit of U.S. Provisional Application Ser. No. 62/848,589 filed 16 May 2019 and of U.S. Provisional Application Ser. No. 62/958,911 filed 9 Jan. 2020 which are included by reference as if fully set-forth herein.

### FIELD OF THE INVENTION

The field of art to which this invention generally pertains is local anesthesia and more specifically to methods for reducing a throat irritation comprising use of transient receptor potential channel modulators and compositions for use in such methods.

### BACKGROUND

Smoking of materials such as cannabis, cannabinoids or tobacco are known to often cause irritation of the throat due to inhalation of vapors or particles of these materials. Known methods of treating such throat irritation include gargling with salt water, sucking on a throat lozenge, sipping on herbal tea with honey and the like, which may not be available to the user at the time of inhalation and are known to have only limited effectivity.

### SUMMARY OF THE INVENTION

According to an aspect of an embodiment of the present invention, provided is a method for reducing a throat irritation caused by inhaling vapors or particles of at least one material selected from the group consisting of cannabis, cannabinoids, tobacco, a herb or combinations thereof, the method comprising administering to a subject in need thereof at least one transient receptor potential (TRP) channel modulator prior to or simultaneously with said inhaling said vapors or particles of said at least one material.

According to a further aspect of an embodiment of the present invention, provided is a composition comprising at least 1 mg of at least one transient receptor potential (TRP) channel modulator and at least one material selected from the group consisting of a cannabinoid, tobacco and combinations thereof, wherein use of said composition has a reduced effect in causing an irritation of the throat as compared to use of a composition comprising a same amount of said material in the absence of said at least one TRP channel modulator.

According to a further aspect of an embodiment of the present invention, provided is a composition for providing pain-selective analgesia via TRP channels activation, comprising (i) at least one TRP channel agonist, and/or at least one TRP channels partial agonist, and (ii) optionally at least one local anesthetic.

### DETAILED DESCRIPTION OF THE INVENTION

The present invention, in at least some embodiments thereof, relates to methods for reducing a throat irritation caused by inhaling vapors or particles of cannabis, cannabinoids, tobacco, a herb or combinations thereof by administering at least one transient receptor potential (TRP) channel modulator prior to or simultaneously with inhaling said vapors or particles.

Unless indicated otherwise, the term “local anesthetic” refers to a medication that causes absence of pain sensation. Local anesthetic as used herein refers to medication applied via administration methods including topical anesthesia, field block, ring block, local infiltration and nerve block.

As used herein, the term “throat irritation” refers to dryness, soreness, pain or inflammation of the throat.

As used herein, the term “reducing a throat irritation” refers to reducing the severity, duration or frequency of occurrence of a throat irritation by at least 20% as compared to that which occurs in the absence of use of a method as disclosed herein. According to some embodiments, severity of a throat irritation is determined by a subject assigning a value on a scale of 1 to 5 to the level of pain associated with the condition.

As used herein, in some embodiments the term “vapor” refers to a substance in a gas phase obtained by heating a material such as cannabis, cannabinoid, tobacco or a herb to a temperature just below the combustion point of the material. As used herein, in some embodiments the term “vapor” refers to a substance in a gas phase obtained by heating a material such as cannabis, cannabinoid, tobacco or a herb to a temperature above the boiling point of the material. In some such embodiments, when the material is cannabis, the material is heated to a temperature of between 180° and 500° C.

As used herein, in some embodiments the term “particles” refers to particles of a material such as cannabis, cannabinoid, tobacco or a herb present in smoke obtained by burning the material or present in the form of an aerosol. Hence in some such embodiments, inhaling of the particles is achieved by smoking the material. As used herein, in some embodiments the term “particles” refers to particles of a material such as cannabis, cannabinoid, tobacco or a herb in arousal obtained by spraying the extract of the material.

As used herein, the term “TRP channel modulator” refers to an agent which increases or decreases TRP channel function, such as an agonist, antagonist, partial agonist or an allosteric modulator of TRP channel function.

As used herein, the term “administering” includes any mode of administration, such as oral, subcutaneous, sublingual, transmucosal, parenteral, intravenous, intra-arterial, buccal, sublingual, topical, vaginal, rectal, ophthalmic, otic, nasal, inhaled, intramuscular, intraosseous, intrathecal, and transdermal, or combinations thereof.

Unless indicated otherwise, the term “cannabinoid” as used herein refers to a compound that affects the endocannabinoid system. Cannabinoids are agonists or antagonists to receptors of the endocannabinoid system.

As used herein, the term “THC” refers to THCa (tetrahydrocannabiniolic acid) and/or to THC (tetrahydrocannabiniol) unless indicated otherwise. As used herein, the term “CBD” refers to CBDa (cannabidiolic acid) and/or to CBD (cannabidiol) unless indicated otherwise. As used herein, the term “CBG” refers to CBGa (cannabigerolic acid) and/or to CBG (cannabigerol) unless indicated otherwise. As used herein, the term “CBN” refers to CBNa (Cannabinolic acid) and/or to CBN (cannabinol) unless indicated otherwise. As used herein, the term “CBC” refers to CBCa (cannabichromenic acid) and/or to CBC (Cannabichromene) unless indicated otherwise. As used herein, the term “CBL” refers to CBLa (Cannabicycol acid) and/or to CBL (Cannabicyclol) unless indicated otherwise. As used herein, the term “THCV” refers to THCVa (tetrahydrocannabivarin acid) and/or to THCV (tetrahydrocannabivarin) unless indicated otherwise. As used herein, the term “CBDV” refers to CBDVa (cannabigerovarin acid) and/or to CBDV (cannabidivarin) unless indicated otherwise.

As used herein, “terpene” refers to both terpenes and terpenoids.

The particulars shown herein are by way of example and for purposes of illustrative discussion of the various embodiments of the present invention only and are presented in the cause of providing what is believed to be the most useful and readily understood description of the principles and conceptual aspects of the invention. In this regard, no attempt is made to show details of the invention in more detail than is necessary for a fundamental understanding of the invention, the description making apparent to those skilled in the art how the several forms of the invention may be embodied in practice.

The present invention will now be described by reference to more detailed embodiments. This invention may, however, be embodied in different forms and should not be construed as limited to the embodiments set forth herein. Rather, these embodiments are provided so that this disclosure will be thorough and complete, and will fully convey the scope of the invention to those skilled in the art.

Unless otherwise defined, all technical and scientific terms used herein have the same meaning as commonly understood by one of ordinary skill in the art to which this invention belongs. The terminology used in the description of the invention herein is for describing particular embodiments only and is not intended to be limiting of the invention. As used in the description of the invention and the appended claims, the singular forms “a,” “an,” and “the” are intended to include the plural forms as well, unless the context clearly indicates otherwise.

Unless otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in the following specification and attached claims are approximations that may vary depending upon the desired properties sought to be obtained by the present invention. At the very least, and not as an attempt to limit the application of the doctrine of equivalents to the scope of the claims, each numerical parameter should be construed in light of the number of significant digits and ordinary rounding approaches.

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements. Every numerical range given throughout this specification will include every narrower numerical range that falls within such broader numerical range, as if such narrower numerical ranges were all expressly written herein.

As used herein, the terms “comprising”, “including”, “having” and grammatical variants thereof are to be taken as specifying the stated features, integers, steps or components but do not preclude the addition of one or more additional features, integers, steps, components or groups thereof. These terms encompass the terms “consisting of” and “consisting essentially of”.

Additional advantages of the invention will be set forth in part in the description which follows, and in part will be obvious from the description, or may be learned by practice of the invention. It is to be understood that both the foregoing general description and the following detailed description are exemplary and explanatory only and are not restrictive of the invention, as claimed.

According to an aspect of an embodiment of the present invention, there is provided a method for reducing a throat irritation caused by inhaling vapors or particles of at least one material selected from the group consisting of cannabis, cannabinoids, tobacco, a herb or combinations thereof, the method comprising administering to a subject in need thereof at least one transient receptor potential (TRP) channel modulator prior to or simultaneously with said inhaling said vapors particles of said at least one material.

According to an aspect of an embodiment of the present invention, there is provided at least one transient receptor potential (TRP) channel modulator for use in reducing a throat irritation caused by inhaling vapors or particles of at least one material selected from the group consisting of cannabis, cannabinoids, tobacco, a herb or combinations thereof, wherein said TRP channel modulator is for administration prior to or simultaneously with said inhaling of said vapors or particles.

According to an embodiment, said at least one TRIP channel modulator is administered as a vapor.

According to an embodiment, said at least one TRP channel modulator is administered in an aerosol.

According to an embodiment, said at least one TRP channel modulator is administered before inhaling said vapors or particles of said material, in some embodiments at least xxx seconds/minutes before inhaling said vapors or particles. According to one such embodiment, the material comprises at least one cannabinoid, wherein at least 0.5 mg of said at least one TRP channel modulator is administered at a time interval of from about 0.2 seconds to about 60 seconds, more preferably from about 0.25 seconds to about 10 seconds before inhaling at least 2 mg of said cannabinoid. In some such embodiments, at least 0.5, at least 1, at least 2, at least 5, at least 10, at least 15, at least 20, at least 25, at least 30, at least 35, at least 40, at least 45 or even at least 50 mg of said one TRP channel modulator are administered before inhaling at least 2 mg of said cannabinoid.

According to an embodiment, said at least one TRP channel modulator is selected from the group consisting of a TRP channel agonist, a TRP channel partial agonist, a TRP channel antagonist and a combination thereof. According to an embodiment, said at least one TRP channel modulator comprises a TRP channel allosteric modulator.

According to an embodiment, said at least one TRP channel modulator is selected from the group consisting of a TRPA1 channel modulator, a TRPV1 channel modulator, and a combination thereof.

According to an embodiment, said at least one TRP channel modulator is selected from the group consisting of Citral, Camphor, Carvacrol, Thymol., Menthol, 1,4-cineole, 1,8-cineole, Geraniol, citronellol, Geranial, Neral, Nerol, Vanillin, Terpineol, Linalool, Perillaldehyde, Perillaketone, Umbellulone, Polygodial, Drimanial, Isovelleral, Cinnamodial, Cinnarnosmolide, Cinnamolide, Warburganal, Scalaradial, Aframodial, Ancistrodial, Merulidial Drimenol, Capsaicin, Capsiate, Dihydrocapsiate, Nordihydrocapsiate, Piperine, Eugenol, Resiniferatoxin, Gingerol, Shogaol, Zingerone, Paradol, Grifolin, Neogrifolin, Albaconol, Scutigeral, Cochinchinenin, Loureirin, Allyl isothiocyanate, Cinnamaldehyde, Allicin, Diallyl. disulphide, Ligustilide, Dehydroligustilide, Alpha-spinastera Guaiacol, Methyl salicylate, Methyl syringate, Resveratrol, Pinosylvin methyl ether, Ginsenoside, Evodiamine, Hydroxy-alpha sanshool, Artepillin, Thapsigargin, Ricinoleic acid, Vanillotoxins, benzaldehyde, Carveol, 6-tert-butyl-m-cresol, Dihydrocarveol, Borneol, Fenchyl alcohol, 2-methylisoborneol and combinations thereof.

According to an embodiment, said at least one TRP channel modulator comprises at least one terpene.

According to an embodiment, said at least one TRP channel modulator is administrated in a delivery form selected from the group consisting of a cigarette, a cigarette filter, a cigarette filter holder, a cigarette folding paper, a medical patch, a tablet, a gel capsule, a topical, a cream, a varnish, an oil, a spray, an edible, a beverage, a vaporizer filer, a vaporizer liquids, and combinations thereof. In some such embodiments, the cigarette filter or cigarette folding paper comprises or consists of an absorbent material into which the TRP channel modulator is absorbed. In some such embodiments the cigarette filter holder is a silicone holder containing therein an absorbent material. The absorbent material maybe, for example, sponge, paper or combinations thereof. In some embodiments, heating of the absorbent material causes release of the TRP channel modulator in a vapor form. In some embodiments, heating of the absorbent material causes release of the TRP channel modulator in aerosol form.

According to an embodiment, the cannabis, cannabinoids, tobacco, a herb or combinations thereof are provided together with the TRP channel modulator in a single product selected from the group consisting of a cigarette ground material, or vaporizer liquid.

In some embodiments, the TRP channel modulator is absorbed at a location on the product which, when in use, would be closer to the mouth of the user than the cannabis, cannabinoids, tobacco, a herb or combinations thereof and therefore inhaled prior to inhalation of the cannabis, cannabinoids, tobacco, a herb or combinations thereof.

According to an embodiment, said at least one TRP channel modulator has a boiling point in the range of from about 100° C. to about 300° C. at 76o mmHg, in some embodiments, less than 220° C. , such as less than 210° C., less than 200° C., less than 190° C., less than 180° C., less than 170° C., less than 160° C., less than 150° C. According to an embodiment, said at least one TRP channel modulator has a boiling point of greater than 1₅0° C. at 76o mmHg, such as greater than 160° C., greater than 170° C., greater than 180° C., greater than 190° C., greater than 200° C., or greater than 210° C.

According to an embodiment, said at least one TRP channel modulator has a partial vapor pressure of higher than 0.1 mmHg at 25° C., such as higher than 0.2, higher than 0.3, higher than 0.4, higher than 0.5, higher than 0.6, higher than 0.7, higher than 0.8, higher than 0.9, higher than 1.0, higher than 1.1, higher than 1.2, higher than 1.3, higher than 1.4, higher than 1.5, higher than 1.6, higher than 1.7, higher than 1.8, higher than 1.9 or even higher than 2.0 mmHg.

According to an embodiment, said at least one TRP channel modulator is provided as a single dose.

According to an embodiment, said at least one TRP channel modulator is provided in an amount ranging between 0.5 mg to 500 mg.

According to a further aspect of some embodiments of the present invention, there is provided a composition comprising at least 1 mg of at least one transient receptor potential (TRP) channel modulator and at least one material selected from the group consisting of a cannabinoid, tobacco and combinations thereof, wherein use of said composition has a reduced effect in causing an irritation of the throat as compared to use of a composition comprising a same amount of said material in the absence of said at least one TRP channel modulator. According to some such embodiments, the composition comprises at least 2, at least 5, at least 10, at least 15, at least 20, at least 25, at least 30, at least 35, at least 4o, at least 45 or at least 50 mg of at least one TRP channel modulator.

According to an embodiment, said at least one TRP channel modulator is volatile.

According to an embodiment, said at least one TRP channel modulator is selected from the group consisting of a TRP channel agonist, a TRP channel partial agonist, a TRP channel antagonist and a combination thereof. According to an embodiment, said at least one TRP channel modulator comprises a TRP channel allosteric modulator.

According to an embodiment, said at least one TRP channel modulator is selected from the group consisting of a TRPA1 channel modulator, a TRPV1 channel modulator, and a combination thereof.

According to an embodiment, said least one TRP channel modulator is selected from the group consisting of Citral, Camphor, Carvacrol, Thymol, Menthol, 1,4-cineole, 1,8-cineole, Geraniol, citronellol, Geraniol, Neral, Nerol, Vanillin, Terpineol, Linalool, Perillaldehyde, Perillaketone, Umbellulone, Polygodial, Drimanial, Isovelleral, Cinnamodial, Cinnamosmolide, Cinnamolide, Warburganal, Scalaradial, Aframodial, Ancistrodial, Merulidial Drimenol, Capsaicin, Capsiate, Dihydrocapsiate, Nordihydrocapsiate, Piperin.e, Eugenol, Resiniferatoxin, Gingerol, Shogaol, Zingerone, Paradol, Grifolin, Neogrifolin, Albaconol, Scutigeral, Cochinchinenin, Loureirin, Allyl isothiocyanate, Cinnamaldehyde, Allicin, Diallyl disulphide, Ligustilide, Dehydroligustilide, Alpha-spinasterol, Guaiacol, Methyl salicylate, Methyl syringate, Resveratrol, Pinosylvin methyl ether, Ginsenoside, Evodiamine, Hydroxy-alpha sanshool, Artepillin, Thapsigargin, Ricinoleic acid, Vanillotoxins, benzaldehyde, Carveol, 6-tert-butyl-m-cresol, Dihydrocarveol, Borneol, Fenchyl alcohol, 2-methylisoborneol and combinations thereof.

According to an embodiment, said cannabinoid is selected from the group consisting of CBD, CBDV, THC, THCV, CBG, CBGV, CBC, CBCV, CBN and combination thereof.

According to an embodiment, provided is a composition for providing pain-selective analgesia via TRP channels activation, comprising (i) at least one TRP channel agonist and/or at least one TRP channels partial agonist, and (ii) optionally at least one local anesthetic in a specific amount.

According to an embodiment, the composition comprises (i) at least one TRP channel agonist and (ii) at least one local anesthetic.

According to an embodiment, the composition comprises (i) at least two TRP channel agonists, such as at least three, or at least four; TRP channel agonists and (ii) at least two local anesthetics, such as at least three, or at least four local anesthetics.

According to an embodiment, the composition comprises (i) at least one TRP channels agonist, (ii) at least one local anesthetic, and (iii) at least one TRP channels partial agonist.

According to an embodiment, the composition comprises (i) at least two TRP channel agonists, such as at least three, or at least four TRP channel agonists; (ii) at least two local anesthetics, such as at least three, or at least four local anesthetics; and (iii) at least two TRP channel partial agonists, such as at least three, or at least four TRP channel partial agonists.

According to an embodiment, the composition comprises (i) at least one TRP channels agonist, and (ii) at least one TRP channels partial agonist.

According to an embodiment, the composition comprises (i) at least two TRP channels agonists, such as at least three, or at least four TRP channel agonists; and (ii) at least two TRP channel partial agonists, such as at least three, or at least four TRP channel partial agonists.

According to an embodiment, the composition comprises at least two TRP channel partial agonists. According to an embodiment, the composition comprises at least three TRP channel partial agonists, such as at least four, at least five, or at least six TRP channel partial agonists.

According to an embodiment, the composition comprises at least two TRP channel partial agonists, such as at least one per at least one TRP channel partial agonists.

According to an embodiment, the composition comprises at least one TRP channel agonist, wherein said at least one TRP channel agonist comprises at least one cannabinoid and/or at least one terpene.

According to an embodiment, the composition comprises at least one TRP channel partial agonist, wherein said at least one TRP channel partial agonist comprises at least one cannabinoid and/or at least one terpene.

According to an embodiment, the composition comprises at least one TRP channel agonist and/or at least one TRP channel partial agonist, wherein each of said TRP channel agonist and said TRP channel partial agonist comprise at least one cannabinoid and/or at least one terpene.

According to an embodiment, the composition comprises at least one TRP channel agonist and/or at least one TRP channel partial agonist. According to an embodiment, said at least one TRP channel agonist and/or said at least one TRP channel partial agonist comprises a total of at least 0.5 mg, at least 1, at least 2, at least 5, at least 10, at least 15, at least 20, at least 25, at least 30, at least 35, at least 40, at least 45 or at least 50 mg TRP channel modulators.

According to an embodiment, the composition comprises at least one cannabinoid selected from the group consisting of N-arachidonoyl dopamine, anandamide, CBD, CBDV, THCV, THC, CBG, CBGV, CBC, CBN and combinations thereof. According to an embodiment, the composition comprises at least two cannabinoids, such as at least three, at least four, or at least five cannabinoids.

According to an embodiment, the composition comprises at least one terpene selected from the group consisting of Citral, Camphor, Carvacrol, Thymol, Menthol, 1,4-cineole, 1,8-cineole, Perillaldehyde, Perillaketone, 1,8-cineole, Citronella, Perillaldehyde, Perillaketone, Linalool, Umbellulone Polygodial, Drimanial, Isovelleral, Cinnamodial, Cinnamosmolide, Cinnamolide, Warburganal, Scalaradial, Aframodial, Ancistrodial, Merulidial Drimenol, Carveol, 6-tert-butyl-m-cresol, Dihydrocarveol, Borneol, Geraniol, Geranial, neral, nerol, vanillin, terpineol Eugenol, Carveol, Fenchyl alcohol, 2-methylisoborneol and combinations thereof. According to an embodiment, the composition comprises at least two terpenes, such as at least three, at least four, or at least five terpenes.

According to an embodiment, the composition comprises at least one cannabinoid selected from the group consisting of anandamide, N-arachidonoyl dopamine, CBD, CBDa, CBC, THC, THCa, CBG, CBC, CBN and combinations thereof, and at least one terpene selected from the group consisting of Citral, Camphor, Carvacrol, Thymol, Menthol, 1,4-cineole, 1,8-cineole, Perillaldehyde, Perillaketone, Umbellulone Drimanial, Isovelleral, Cinnamodial, Cinnamosmolide, Cinnamolide, Warburganal, Scalaradial, Aframodial, Ancistrodial, Merulidial Drimenol, Geraniol, Geranial, Neral, Nerol, Vanillin, Terpineol, Citronellol, Linalool, Borneol, 2-methylisoborneol, fenchyl alcohol and combinations thereof.

According to an embodiment, the composition comprises at least one cannabinoid selected from the group consisting of anandamide, N-arachidonoyl dopamine, CBD, CBDa and combinations thereof, and at least one terpene selected from the group consisting of Citral, Camphor, Gerniaol, Poligodial, Drimanial, Isovelleral, Cinnamodial, Cinnamosmolide, Cinnamolide, Warburganal, Scalaradial, Aframodial, Ancistrodial, Merulidial Drimenol, Geranial, Neral, Nerol, Vanillin, Terpineol, Citronellol and combinations thereof.

According to an embodiment, the composition comprises at least one cannabinoid selected from the group consisting of anandamide, CBD, CBDa, CBC, THC, THCa, CBG, CBC, CBN and combinations thereof, and at least one terpene selected from the group consisting of Citral, Camphor, Carvacrol, Thymol, Menthol, 1,4-cineole, 1,8-cineole, Poligodial, Isovelleral, Umbellulone, Perillaldehyde, Perillaketone, Linalool, Terpineol, Borneol, 2-methylisoborneol, fenchyl alcohol and combinations thereof

According to an embodiment, the composition comprises at least one cannabinoid selected from the group consisting of anandamide, CBD, CBC, THC and combinations thereof, and at least one of Citral and Camphor.

According to an embodiment, the composition comprises at least one local anesthetic, wherein said at least one local anesthetic is ionized at a physiological pH, such as at the pH of blood.

According to an embodiment, the composition comprises at least one local anesthetic, wherein said at least one local anesthetic is an aminoester.

According to an embodiment, the composition comprises at least one local anesthetic, wherein said at least one local anesthetic is an aminoamide.

According to an embodiment, the composition comprises at least one local anesthetic, wherein said at least one local anesthetic is selected from the group consisting of Benzocaine, Chloroprocaine, Cocaine, Cyclomethycaine, Dimethocaine (Larocaine), Piperocaine, Propoxycaine, Procaine (Novocaine), Proparacaine, Tetracaine (Amethocaine) and combination thereof.

According to an embodiment, the composition comprises at least one local anesthetic, wherein said at least one local anesthetic is selected from the group consisting of Articaine, Bupivacaine, Cinchocaine (Dibucaine), Etidocaine, Levobupivacaine, Lidocaine (Lignocaine), Mepivacaine, Prilocaine, Ropivacaine, Trimecaine and combinations thereof.

According to an embodiment, the composition comprises at least one cannabinoid selected from the group consisting of N-arachidonoyl dopamine, anandamide, CBD, CBDV, THCV, THC, CBG, CBGV, CBC, CBN and combination thereof, and at least one terpene selected from the group consisting of Citral, Camphor Carvacrol, Thymol, Menthol, 1,4-cineole, Perillaldehyde, Perillaketone and combination thereof.

According to an embodiment, the composition comprises at least one cannabinoid selected from the group consisting of anandamide, CBD, CBC and combinations thereof, and at least one of Citral and Camphor.

According to an embodiment, the composition comprises at least one terpene selected from the group consisting of pinene, limonene, linalool, caryophyllene, caryophyllene oxide, myrcene, humulene, borneol, eucalyptol, terpineol, nerolidol, phytol, geraniol, bisabolol, camphene, beta-amyrin, thujone, citronellol, pulegone, cycloartenol, cymene, sabinene, carene, terpinene, fenchol, isopulegol, guaiol, phellandrene, eudesmol, ocimene, cardinene, elemene, gurjunene, farnesene, friedelin, carvacrol, eugenol, geranyl acetate, camphor, menthol, iso-menthone, neral, gerial, viridiflorol, germacrene, thymol, Menth-2-en-1-ol, farensol, carotol, myrtenol and combinations thereof.

According to an embodiment, the composition having a therapeutic effect in treating local pain.

According to an embodiment, the composition comprises at least one terpene selected from the group consisting of Citral, Camphor Carvacrol, Thymol, Menthol, cineole, Perillaldehyde, Perillaketone, Polygodial, Drimanial, Isovelleral, Cinnamodial, Cinnamosmolide, Cinnamolide, Warburganal, Scalaradial, Aframodial, Ancistrodial, Merulidial, Drimenol, Umbellulone, Cineole, Carveol, 6-tert -butyl-m-cresol, Dihydrocarveol, Borneol, Perillaldehyde, Perillaketone and combination thereof. According to an embodiment, the composition comprises at least two terpenes, at least three, at least four, or at least five.

According to an embodiment, the composition comprises at least one terpene selected from the group consisting of pinene, limonene, linalool, caryophyllene, caryophyllene oxide, myrcene, humulene, borneol, eucalyptol, terpineol, nerolidol, phytol, geraniol, bisabolol, camphene, beta-amyrin, thujone, citronellol, pulegone, cycloartenol, cymene, sabinene, carene, terpinene, fenchol, isopulegol, guaiol, phellandrene, eudesmol, ocimene, cardinene, elemene, gurjunene, farnesene, friedelin, carvacrol, eugenol, geranyl acetate, camphor, menthol, iso-menthone, neral, gerial, viridiflorol, germacrene, thymol, Menth-2-en-1-ol, farensol, carotol, myrtenol and combinations thereof. According to an embodiment, the composition comprises at least two terpenes, at least three, at least four, or at least five.

According to an embodiment, the composition comprises at least one compound selected from the groups of Capsaicin, Capsiate, Dihydrocapsiate, Nordihydrocapsiate, Piperine, Eugenol, Resiniferatoxin, Gingerol, Shogaol, Zingerone, Paradol, Grifolin, Neogrifolin, Albaconol, Scutigeral, Cochinchinenin, Loureirin, Allyl isothiocyanate, Cinnamaldehyde, Allicin, Diallyl disulphide, Ligustilide, Dehydroligustilide, Alpha-spinasterol, Guaiacol, Methyl salicylate, Vanillin, Methyl syringate, Resveratrol, Pinosylvin methyl ether, Salidroside, Bisandrographolide, Ginsenoside, Triptolide, Evodiamine, Nicotine, Yohimbine, Hydroxy-alphasanshool, Artepillin, Thapsigargin, Ricinoleic acid, Incensole acetate, Hyperforin, Vanillotoxins, Quinine, Waixenicin, citral, benzaldehyde and combinations thereof. According to an embodiment, the composition comprises at least two compounds, such as at least three, at least four, or at least five compounds.

According to an embodiment, provided is a product comprising said composition, wherein the product, is selected from the group consisting of tablets, gel capsules, medical patches, topicals, creams, varnishes, sublingual oils, sprays, edibles, beverages, suppositories, tampons, rectal candles, cigarettes vaporizer liquids, nasal preparations, preparations containing micro and/or nano-emulsions, preparations containing micro and/or nano-particles and combinations thereof.

According to an embodiment, the composition comprises at least one agonist, at least one partial agonist, or both, wherein said at least one partial agonist and/or said agonist are selected from the group consisting of Citral, Camphor, Carvacrol, Thymol, Menthol, 1,4-cineole, 1,8-cineole, geraniol, citronellol, geranial, neral, nerol, vanillin, terpineol, Perillaldehyde, Perillaketone, Umbellulone, Polygodial, Drimanial, Isovelleral, Cinnamodial, Cinnamosmolide, Cinnamolide, Warburganal, Scalaradial, Aframodial, Ancistrodial, Merulidial Drimenol, Capsaicin, Capsiate, Dihydrocapsiate, Nordihydrocapsiate, Piperine, Eugenol, Resiniferatoxin, Gingerol, Shogaol, Zingerone, Paradol, Grifolin, Neogrifolin, Albaconol, Scutigeral, Cochinchinenin, Loureirin, Allyl isothiocyanate, Cinnamaldehyde, Allicin, Diallyl disulphide, Ligustilide, Dehydroligustilide, Alpha-spinasterol, Guaiacol, Methyl salicylate, Methyl syringate, Resveratrol, Pinosylvin methyl ether, Ginsenoside, Evodiamine, Hydroxy-alpha sanshool, Artepillin, Thapsigargin, Ricinoleic acid, Vanillotoxins, benzaldehyde and combination thereof.

According to an embodiment, provided is a product comprising said composition, wherein the product is selected from the group consisting of a tablets, gel capsules, medical patches, topicals, creams, varnishes, sublingual oils, sprays, edibles, beverages, suppositories, tampons, rectal candles, cigarettes, vaporizer liquids, nasal preparations, preparations containing micro and/or nano-emulsions, preparations containing micro and/or nano-particles, cigarette filter, a cigarette filter holder, a cigarette folding paper, a topical, a varnish, a spray and combination thereof. According to a preferred embodiment, the product is selected from the group consisting of a cigarette filter, a cigarette filter holder, a cigarette folding paper and combination thereof

According to an embodiment, the composition comprises at least 2% by weight carrier, at least 3%, at least 5%, at least 10%, at least 15%, at least 20%, at least 25%, at least 30%, at least 35% or at least 40% by weight.

According to a further aspect of an embodiment of the present invention, provided is a method of providing pain-selective analgesia via TRP channels activation comprising administering to a subject in need thereof a composition comprising at least one TRP channel agonist, and/or at least one TRP channel partial agonist.

According to a further aspect of an embodiment of the present invention, provided is a composition comprising at least one TRP channel agonist, and/or at least one TRP channel partial agonist for use in providing pain-selective analgesia via TRP channel activation.

According to a further aspect of an embodiment of the present invention, provided is a method of treating local pain comprising administering to a subject in need thereof a composition comprising at least one TRP channel agonist, and/or at least one TRP channel partial agonist.

According to a further aspect of an embodiment of the present invention, provided is a composition comprising at least one TRP channel agonist, and/or at least one TRP channel partial agonist for use in treating local pain.

According to a further aspect of an embodiment of the present invention, provided is a method of treating migraine and migraine-related symptoms, comprising administering to a subject in need thereof a composition at least one TRP channel agonist, and/or at least one TRP channel partial agonist.

According to a further aspect of an embodiment of the present invention, provided is a composition comprising at least one TRP channel agonist, and/or at least one TRP channel partial agonist for use in treating migraine or symptoms thereof.

According to a further aspect of an embodiment of the present invention, provided is a method of treating osteoporosis and osteoporosis-related symptoms comprising administering to a subject in need thereof a composition at least one TRP channel agonist, and/or at least one TRP channel partial agonist.

According to a further aspect of an embodiment of the present invention, provided is a composition comprising at least one TRP channel agonist, and/or at least one TRP channel partial agonist for use in treating osteoporosis or symptoms thereof.

### EXAMPLES

### Example 1: Reducing a Throat Irritation caused by Smoking a Cigarette comprising 20% THC.

3 mg of a TRP channel modulator comprising a mixture of Citral and Camphor in liquid form is absorbed in a filter embedded within a modulator holding element of an inhalation device such as disclosed in FIG. 1 of PCT Publication No. WO/2019/75808. A cigarette 200 mg of THC is attached to the inhalation device. A subject smokes the cigarette, whereupon a vapor stream from the cigarette passes through the filter, such that the heat from the vapor stream causes release of the absorbed TRP channel modulator into the vapor stream. Due to the relative volatilities of the THC and the TRP channel modulator, an amount of 0.5 mg of the TRP channel modulator reaches the throat of the subject prior to 2 mg of THC.

### Example 2: Reducing a Throat Irritation caused by Inhaling Cannabis Plant Material comprising 10% THC and 10% CBD.

A TRP channel modulator comprising a mixture of Thymol, Menthol and 1,8-cineole is absorbed in a vaporizer filter embedded within a modulator holding element of an inhalation device such as disclosed in FIG. 3 of PCT Publication No. WO/2019/175808 connected to a vaporizer. A cannabis plant material comprising 10% THC and 10% CBD is vaporized in the vaporizer. A subject inhales through the mouthpiece of the inhalation device, whereupon a vapor stream from the vaporized cannabis plant material passes through the filter, such that the heat from the vapor stream causes release of the absorbed TRP channel modulator into the vapor stream. Due to the relative volatilities of the cannabis plant material and the TRP channel modulator, an amount of f 1 mg of the TRP channel modulator reaches the throat of the subject prior to inhalation of plant material comprising 1 mg of THC and 1 mg of CBD.

### Example 3: Reducing a Throat Irritation caused by Smoking a Cigarette comprising 15% THC, 3% CBD and 2% CBN.

A TRP channel modulator comprising Carvacrol is absorbed in a cigarette folding paper enclosing a cigarette comprising 15% THC, 3% CBD and 2% CBN. A subject smokes the cigarette whereupon a vapor stream is formed, such that heat from the vapor stream causes release of the absorbed TRP channel modulator into the vapor stream such that the THC, CBD and CBN reach the throat of the subject simultaneously with the TRP channel modulator.

Example 4: Reducing a throat irritation caused by smoking a cigarette comprising 5% THC, 10% CBD and 2% CBC.

A TRP channel modulator comprising 15 g of a combination of Terpineol and Linalool is administered to a subject in the form of an aerosol spray squirted to the throat. The subject than smokes a cigarette comprising cannabinoids (5% THC, 10% CBD and 2% CBC). An amount of1.5 mg of a mixture of Terpineol and Linalool reaches the throat before inhaling 1.5 mg of the cannabinoids.

### Example 5: A composition for Reducing a Throat Irritation

A cigarette comprising 2 mg of a mixture of Borneol, Citral and fenchyl alcohol and 12 mg of tobacco.

### Example 6: A Composition for Reducing a Throat irritation—

A cigarette comprising 3 mg of a mixture of Terpineol and Cinnamaldehyde and 8 mg of THC and ₄ mg of CBD.

Examples—compositions for providing pain-selective analgesia

| | TRP channel agonist or partial | Local |
|---|---|---|
| Delivery form | agonist | anesthetic |
| Topical | A mixture of CBD, Carvacrol and | Benzocaine |
| | Linalool | |
| medical patch | Thymol | Procaine |
| Cream | A mixture of Cinnamosmolide, | Piperocaine |
| | Cinnamolide and Linalool | |
| Gel capsule | A mixture of THC, CBC and Borneol | |
| Tablet | A mixture of CBG, Eugenol and Geranial | |
| varnished | Vanillin | Tetracaine |
| Spray | A mixture of Citral, Camphor and Neral. | |
| sublingual oil | A mixture of CBG and 1,8-cineole | |

1. A method for reducing a throat irritation caused by inhaling vapors or particles of at least one material selected from the group consisting of cannabis, cannabinoids, tobacc0, a herb or combinations thereof, the method comprising administering to a subject in need thereof at least one transient receptor potential (TRP) channel modulator, optionally as a vapor, prior to or simultaneously with said inhaling said vapors or particles of said at least one material.

2. (canceled)

3. The method of claim 1, wherein said material comprises cannabinoid, wherein at least 0.5 mg of said at least one TRP channel modulator is administered before inhaling 2 mg of said cannabinoid.

4. The method of claim 1, wherein said at least one TRP channel modulator is selected from the group consisting of a TRP channel agonist, a TRP channel partial agonist, a TRP channel antagonist, TRPA1 channel modulator, a TRPV1 channel modulator and a combination thereof.

5. (canceled)

6. The method of claim 1, wherein said at least one TRP channel modulator is selected from the group consisting of Citral, Camphor, Carvacrol, Thymol, Menthol, 1,4-cineole, Geraniol, citronellol, Geranial, Neral, Nerol, Vanillin, Terpineol, Linalool, Perillaldehyde, Perillaketone, Umbellulone, Polygodial, Drimanial, Isovelleral, Cinnamodial, Cinnamosmolide, Cinnamolide, Warburganal, Scalaradial, Aframodial, Ancistrodial, Merulidial Drimenol, Capsaicin, Capsiate, Dihydrocapsiate, Nordihydrocapsiate, Piperine, Eugenol, Resiniferatoxin, Gingerol, Shogaol, Zingerone, Paradol, Grifolin, Neogrifolin, Albaconol, Scutigeral, Cochinchinenin, Loureirin Allyl isothiocyanate, Cinnamaldehyde, Allicin, Diallyl disulphide, Ligustilide, Dehydroligustilide, Alpha-spinasterol, Guaiacol, Methyl salicylate, Methyl syringate, Resveratrol, Pinosylvin methyl ether, Ginsenoside, Evodiamine, Hydroxy-alpha sanshool, Astepillin, Thapsigargin, Ricinoleic acid, Vanillotoxins, benzaldehyde, Carveol, 6-tert-butyl-m-cresol, Dihydrocarveol, Borneol, Fenchyl alcohol, 2-methylisoborneol and combinations thereof.

7. (canceled)

8. The method of claim 1, wherein said at least one TRP channel modulator is administrated in a delivery form selected from the group consisting of a cigarette, a cigarette filter, a cigarette filter holder, a cigarette folding paper, a medical patch, a tablet, a gel capsule, a topical, a cream, a varnish, an oil, an aerosol, a spray, an edible, a beverage, a vaporizer filer, a vaporizer liquids, and combinations thereof.

9. The method of claim 1, wherein said at least one TRP channel modulator has a boiling point of less than 220° C. at 760 mmHg, has a partial vapor pressure of higher than 0.1 at mmHg at 25° C. or both.

10-11. (cancelled)

12. A composition comprising at least 1 mg of at least one transient receptor potential (TRP) channel modulator and at least one material selected from the group consisting of a cannabinoid, tobacco and combinations thereof, wherein use of said composition has a reduced effect in causing an irritation of the throat as compared to use of a composition comprising a same amount of said material in the absence of said at least one TRP channel modulator.

13. (canceled)

14. The composition of claim 12, wherein said at least one TRP channel modulator is selected from the group consisting of a TRP channel agonist, a TRP channel partial agonist, a TRP channel antagonist, TRPA1 channel modulator, a TRPV1 channel modulator and a combination thereof.

15. (canceled)

16. The Composition of claim 12, wherein said at least one TRP channel modulator is selected from the group consisting of Citral, Camphor, Carvacrol, Thymol, Menthol, 1,4-cineole, Geraniol, citronellol, Geranial, Neral, Nerol, Terpineol, Linalool, Perillaldehyde, Perillaketone, Umbellulone, Polygodial Drimanial, Isovelleral, Cinnamodial, Cinnamosmolide, Cinnamolide, Warburganal, Scalaradial, Aframodial, Ancistrodial, Merulidial Drimenol, Capsaicin, Capsiate, Dihydrocapsiate, Nordihydrocapsiate, Piperine, Eugenol, Resiniferatoxin, Gingerol, Shogaol, Zingerone, Paradol, Grifolin, Neogrifolin, Albaconol, Scutigeral, Cochinchinenin, Loureirin, Allyl isothiocyanate, Cinnamaldehyde, Ailicin. Diallyl disulphide, Ligustilide, Dehydroligustilide, Alpha-spinasterol, Guaiacol, Methyl salicylate, Methyl syringate, Resveratrol, Pinosylvin methyl ether, Ginsenoside, Evodiamine, Hydroxy-alpha sanshool, Artepillin, Thapsigargin, Ricinoleic acid, Vanillotoxins, benzaldehyde, Carveol, 6-tert-butyl-m-cresol, Dihydrocarveol, Borneol, Fenchyl alcohol, 2-methylisoborneol and combinations thereof.

17. (canceled)

18. A composition for providing pain-selective analgesia via TRP channels activation, comprising (i) at least one TRP channel agonist, and/or at least one TRP channel partial agonist, and (ii) optionally at least one local anesthetic.

19-22. (canceled)

23. The composition of claim 18, wherein said at least one TRP channel agonist and/or said at least one partial agonist is selected from the group consisting of at least one cannabinoid, at least one terpene and a combination thereof.

24-26. (canceled)

27. The composition of claim 18, comprising at least one terpene selected from the group consisting of Citral, Camphor, Carvacrol, Thymol, Menthol, 1,4-cineole, 1,8-cineole, Citronellol, Perillaldehyde, Perillaketone, Linalool, Umbellulone Polygodial, Drimanial, Isovelleral, Cinnamodial, Cinnamosmolide, Cinnamolide, Warburganal, Scalaradial, Aframodial, Ancistrodial, Merulidial Drimenol, Carveol, 6-tert-butyl-m-cresol, Dihydrocarveol, Borneol, Geraniol, Geranial, neral, nerol, vanillin, terpineol, Eugenol, Carveol, Fenchyl alcohol, 2-methylisoborneol, pinene, limonene, caryophyllene, caryophyllene oxide, myrcene, humulene, eucalyptol, nerolidol, phytol, bisabolol, camphene, beta-amyrin, thuj one, cycelempulegone, cycloartenol, cymene, sabinene, carene, terpinene, fenchol, isopulegol, guaiol, phellandrene, eudesmol, ocimene, cardinene, elemene, gurjunene, farnesene, friedelin, carvacrol, geranyl acetate, iso-menthone, gerial, viridiflorol, germacrene, Menth-2-en-1-ol, farensol, carotol, myrtenol and combinations thereof.

28-33. (canceled)

34. The composition of claim 18, wherein said at least one local anesthetic is ionized at physiological pHand/or is selected from the group consisting of aminoesters, aminoamides, Benzocaine, Chloroprocaine, Cocaine, Cyclomethycaine, Dimethocaine (Larocaine), Piperocaine, Propoxycaine, Procaine (Novocaine), Proparacaine, Tetracaine (Amethocaine), Articaine, Bupivacaine, Cinchocaine (Dibucaine), Etidocaine, Levobupivacaine, Lidocaine (Lignocaine), Mepivacaine, Prilocaine, Ropivacaine, Trimecaine and combination thereof.

35-36. (canceled)

37. The composition of claim 18, comprising a compound selected from the group consisting of Capsaicin, Capsiate, Dihydrocapsiate, Nordihydrocapsiate, Piperine, Eugenol, Resiniferatoxin, Gingerol, Shogaol, Zingerone, Paradol, Grifolin, Neogrifolin, Albaconol, Scutigeral, Cochinchinenin, Loureirin, Allyl isothiocyanate, Cinnamaldehyde, Allicin, Diallyl disulphide, Ligustilide, Dehydroligustilide, Alpha-spinasterol, Guaiacol, Methyl salicylate, Vanillin, Methyl syringate, Resveratrol, Pinosylvin methyl ether, Salidroside, Bisandrographolide, Ginsenoside, Triptolide, Evodiamine, Nicotine, Yohimbine, Hydroxy-alphasanshool, Artepillin, Thapsigargin, Ricinoleic acid, Incensole acetate, Hyperforin, Vanillotoxins, Quinine, Waixenicin, citral, benzaldehyde, and combinations thereof.

38. The composition of claim 18, wherein said at least one agonist and/or said at least one partial agonist are selected for the group consisting of Citral, Camphor, Carvacrol, Thymol, Menthol, 1,4-cineole, 1,8-cineole, geraniol, citronellol, geranial, neral, nerol, vanillin, terpineol, Perillaldehyde, Perillaketone, Umbellulone, Polygodial, Drimanial, Isovelleral, Cinnamodial, Cinnamosmolide, Cinnamolide, Warburganal, Scalaradial, Aframodial, Ancistrodial, Merulidial Drimenol, Capsaicin, Capsiate, Dihydrocapsiate, Nordihydrocapsiate, Piperine, Eugenol, Resiniferatoxin, Gingerol, Shogaol, Zingerone, Paradol, Grifolin, Neogrifolin, Albaconol, Scutigeral, Cochinchinenin, Loureirin, Ally isothiocyanate, Cinnamaldehyde, Allicin, Diallyl disulphide, Ligustilide, Dehydroligustilide, Alpha-spinasterol, Guaiacol, Methyl salicylate, Methyl syringate, Resveratrol, Pinosylvin methyl ether, Ginsenoside, Evodiamine, Hydroxy-alpha sanshool, Artepillin, Thapsigargin, Ricinoleic acid, Vanillotoxins, benzaldehyde and combination thereof.

39. A product comprising a composition according to claim 18, wherein said product is selected from the group consisting of tablets, gel capsules, medical patches, topicals, creams, varnishes, sublingual oils, aerosols, sprays, edibles, beverages, suppositories, tampons, rectal candles, cigarettes, vaporizer liquids, nasal preparations, preparations containing micro and/or nano-emulsions, a cigarette filter, a cigarette filter holder, a cigarette folding paper, a topical, a varnish, a spray, a cigarette filter, a cigarette filter holder, a cigarette folding paper and combination thereof

40. (canceled)

41. A method of providing pain-selective analgesia via TRP channels activation comprising administering to a subject in need thereof a composition or a product according to claim 18.

42. A method of treating local pain comprising administering to a subject in need thereof a composition or a product according to claim 18.

43. A method of treating migraine and migraine-related symptoms comprising administering to a subject in need thereof a composition or a product according to claim 18

44. A method of treating osteoporosis and osteoporosis-related symptoms comprising administering to a subject in need thereof a composition or a product according to claim 18.
